(19) United States
(12) Patent Application Publication
(10) Pub. No.: US 2026/0294782 A1
(43) Pub. Date: Oct. 1, 2026

(54) ARTIFICIAL NAIL COMPOSITION AND ARTIFICIAL NAIL

(71) Applicant: SHOFU INC., Kyoto-shi (JP)

(72) Inventor: Shun SHIMOSOYAMA, Kyoto-shi (JP)

(73) Assignee: SHOFU INC., Kyoto-shi (JP)

(21) Appl. No.: 19/577,728

(22) Filed: Mar. 25, 2026

(30) Foreign Application Priority Data
Mar. 26, 2025 (JP) 2025-052625
Feb. 19, 2026 (JP) 2026-025669

(51) Int. Cl. A61K 8/87; A61Q 3/02

(52) U.S. Cl. CPC A61K 8/87; A61Q 3/02

(57) ABSTRACT

An object of the present invention is to provide an artificial nail composition having adhesion durability to enable putting-on to be maintained for a certain period of time without any problem in daily life and excellent removability to curb damage to a nail at the time of removal. An artificial nail composition of the present invention contains: a component (A) that is a polyurethane resin; a component (B) that is a polymerizable compound having at least two polymerizable functional groups in one molecule; and a component (C) that is a polymerization initiator.

### FIELD OF INVENTION

The present invention relates to an artificial nail composition and an artificial nail.

### BACKGROUND

Artificial nails such as nail polish, gel nails, or acrylic nails are characterized in that durability is good when the artificial nails are applied to human nails, and an aesthetic appearance can be maintained for several weeks. Among the artificial nails, the gel nails have been widely used in recent years since the gel nails are easy to handle and enable various arts to be performed. The gel nails are generally removed using a dedicated machine such as a rotary cutter or a solvent such as acetone since resin coatings on nails deteriorate by abrasion or partial chipping after a certain period of time from a procedure. However, methods using the rotary cutter have to prepare a dedicated device and an advanced technique, and methods using the solvent such as acetone have an adverse effect of degreasing nails or finger- or toe-tip skin. In such removal methods, since there is a high concern that healthy nails or finger- or toe-tip skin may be unnecessarily damaged, there is a demand for an artificial nail composition that can be easily removed by anyone without damaging nails or finger- or toe-tip skin.

As an attempt to solve the above-described problems, a peel-off type artificial nail composition that can be removed by taking off a resin coating like peeling off a seal without using a special machine or a solvent has been proposed. However, the peel-off type artificial nail composition tends to have lower adhesiveness to a nail or lower durability of a resin coating as compared with a conventional artificial nail composition that is removed using a dedicated machine or a solvent, and it has been difficult to enable the peel-off type artificial nail composition to be put on for a certain period of time without any problem in daily life and to be removed without damaging a nail or surrounding tissue. An artificial nail composition exemplified in JP 2024-1559 A is known as an artificial nail composition provided in an attempt to solve such problems.

### SUMMARY OF INVENTION

However, while the artificial nail composition of JP 2024-1559 A has excellent removability to curb damage to a nail at the time of removal, not only abrasion or partial breaking of the resin coating occur early in daily life, but also the resin coating may be peeled off from the nail. Hence, the removability, adhesiveness to a nail, and durability of the resin coating are not all satisfied, and there is room for improvement in the artificial nail composition.

The present invention has been made in view of the above-described circumstances, and an object of the present invention is to provide an artificial nail composition having adhesion durability to enable putting-on to be maintained for a certain period of time without any problem in daily life and excellent removability to curb damage to a nail at the time of removal.

Means for solving the above-described problems include the following aspects.
<1> An artificial nail composition comprising:
   a component (A) that is a polyurethane resin;
   a component (B) that is a polymerizable compound having at least two polymerizable functional groups in one molecule; and
   a component (C) that is a polymerization initiator.
<2> The artificial nail composition according to <1>, wherein the component (A) that is a polyurethane resin has a polyether skeleton.
<3> The artificial nail composition according to <1> or <2>, wherein a weight-average molecular weight (Mw) of the component (A) that is a polyurethane resin is 5000 or more.
<4> The artificial nail composition according to any one of <1> to <3>, wherein the component (B) that is a polymerizable compound having at least two polymerizable functional groups in one molecule has at least one selected from the group consisting of a (meth)acryloyl group, a (meth)acryloyloxy group, and an isoprenyl group.
<5> The artificial nail composition according to any one of <1> to <4>, wherein the component (B) that is a polymerizable compound having at least two polymerizable functional groups in one molecule is a compound having two or three polymerizable functional groups in one molecule.
<6> The artificial nail composition according to any one of <1> to <5>, wherein the component (C) that is a polymerization initiator is a photopolymerization initiator.
<7> The artificial nail composition according to any one of <1> to <5>, wherein the component (C) that is a polymerization initiator is at least one selected from the group consisting of α-hydroxyalkylphenones and acylphosphine oxides.
<8> The artificial nail composition according to any one of <1> to <7>, further comprising a component (D) that is a polymerizable compound excluding the component (B).
<9> The artificial nail composition according to <8>, wherein the component (D) that is a polymerizable compound has, in one molecule, at least one selected from the group consisting of a hydroxyl group and an ether skeleton.
<10> The artificial nail composition according to any one of <1> to <7>, wherein the artificial nail composition contains, with respect to a total amount of the composition,
   5 to 70 mass % of the component (A) that is a polyurethane resin,
   0.01 to 15 mass % of the component (B) that is a polymerizable compound having at least two polymerizable functional groups in one molecule, and
   0.1 to 15 mass % of the component (C) that is a polymerization initiator.
<11> The artificial nail composition according to <8> or <9>, wherein the artificial nail composition contains, with respect to a total amount of the composition,
   5 to 70 mass % of the component (A) that is a polyurethane resin,
   0.01 to 15 mass % of the component (B) that is a polymerizable compound having at least two polymerizable functional groups in one molecule,
   0.1 to 15 mass % of the component (C) that is a polymerization initiator, and
   20 to 90 mass % of the component (D) that is a polymerizable compound excluding the component (B).
<12> The artificial nail composition according to any one of <1> to <11>, wherein the artificial nail composition has a tensile strength of 20 MPa or less when cured.
<13> The artificial nail composition according to any one of <1> to <12>, wherein the artificial nail composition has a breaking strain of 30% or more when cured.
<14> The artificial nail composition according to any one of <1> to <13>, wherein the artificial nail composition has a loss tangent of 0.01 or more when cured.
<15> The artificial nail composition according to any one of <1> to <14>, wherein the artificial nail composition has a glass transition temperature of 100° C. or lower when cured.
<16> An artificial nail obtained from the artificial nail composition according to any one of <1> to <15>.
<17> The artificial nail according to <16>, wherein the artificial nail has a tensile strength of 20 MPa or less.
<18> The artificial nail according to <16> or <17>, wherein the artificial nail has a breaking strain of 30% or more.
<19> The artificial nail according to any one of <16> to <18>, wherein the artificial nail has a loss tangent of 0.01 or more.
<20> The artificial nail according to one of <16> to <19>, wherein the artificial nail has a glass transition temperature of 100° C. or lower.

According to the present invention, it is possible to provide an artificial nail composition having adhesion durability to enable putting-on to be maintained for a certain period of time without any problem in daily life and excellent removability to curb damage to a nail at the time of removal.

### DETAILED DESCRIPTION OF THE PREFERRED EMBODIMENTS

An artificial nail composition of the present invention contains: a component (A) that is a polyurethane resin; a component (B) that is a polymerizable compound having at least two polymerizable functional groups in one molecule; and a component (C) that is a polymerization initiator.

### [(A) Polyurethane Resin]

The component (A) that is a polyurethane resin in the artificial nail composition of the present invention is a substance that has at least one urethane bond unit in a molecular structure and does not have an ethylenically unsaturated double bond as a polymerizable functional group that starts a reaction by applying external energy such as light or heat to form a polymer, and the component (A) can be used without any limitation as long as it is compatible with other components. The component (A) imparts adhesion durability and removability to the artificial nail composition of the present invention. A manufacturing method of the component (A) is not particularly limited, and the component (A) can be obtained, for example, by causing a polyisocyanate having two or more isocyanate groups to react with a polyol having two or more hydroxy groups.

Specific examples of the polyisocyanate include hexamethylene diisocyanate, 2,2,4-trimethylhexamethylene diisocyanate, 2,4,4-trimethylhexamethylene diisocyanate, 2,4-diisocyanate-1-methylcyclohexane, diisocyanate cyclobutane, tetramethylene diisocyanate, o-, m-, or p-xylylene diisocyanate, hydrogenated xylylene diisocyanate, dicyclohexylmethane diisocyanate, dimethyldicyclohexylmethane diisocyanate, lysine diisocyanate, cyclohexane diisocyanate, dodecane diisocyanate, tetramethylxylene diisocyanate, isophorone diisocyanate, tolylene-2,4-diisocyanate, tolylene-2,6-diisocyanate, diphenylmethane-4,4′-diisocyanate, 3,3′-dimethyldiphenylmethane-4,4′-diisocyanate, m- or p-phenylene diisocyanate, chlorophenylene-2,4-diisocyanate, naphthalene-1,5-diisocyanate, diphenyl-4,4′-diisocyanate, carbodiimide-modified diphenylmethane diisocyanate, polyphenyl polymethylene isocyanate, and diphenyl ether diisocyanate, and the examples thereof are not limited thereto. In addition, these polyisocyanates may be used singly or in combination of two or more kinds thereof.

Specific examples of the polyol include ethylene glycol, propylene glycol, 1,3-propanediol, 1,4-butanediol, 1,3-butanediol, 1,5-pentanediol, neopentyl glycol, 1,6-hexanediol, 3-methyl-1,5-pentanediol, 1,4-bis(hydroxymethyl)cyclohexane, bisphenol A, hydrogenated bisphenol A, hydroxypivalyl hydroxypivalate, trimethylolethane, trimethylolpropane, 2,2,4-trimethyl-1,3-pentanediol, glycerin, hexanetriol, polyether glycol; polyoxyethylene glycol, polyoxypropylene glycol, polyoxyethylene polyoxytetramethylene glycol, polyoxypropylene polyoxytetramethylene glycol, and polyoxyethylene polyoxypropylene polyoxytetramethylene glycol, polyether polyols obtained by ring-opening polymerization of these polyhydric alcohols with a cyclic ether bond-containing compound, polyester polyols obtained by dehydration polycondensation of polyhydric alcohols with a polyvalent carboxylic acid, and polycarbonate polyols obtained by transesterification reaction of polyhydric alcohols with a carbonate compound, and the examples thereof are not limited thereto. In addition, these polyols may be used singly or in combination of two or more kinds thereof. Among these polyols, a polyol having a polyether skeleton is preferably used from the viewpoint of improving adhesiveness to a nail.

A polymerization method of the component (A) is not particularly limited. Specific examples of the polymerization method include bulk polymerization and solution polymerization. In addition, a polymerization degree of the component (A) is also not particularly limited and may be a polymer or an oligomer. Note that the term, oligomer, in this specification means a polymer obtained by polymerizing two to several tens of polymerizable monomers. In addition, the term, polymer, means a polymer obtained by polymerizing several tens or more polymerizable monomers and means a polymer other than oligomers.

A form of the component (A) is not particularly limited as long as the component (A) can be compatible with another component, and the component (A) can have any form such as a liquid form, a gel form, a powder form, or a lump form. Among the forms, from the viewpoint of being easily compatible with another component, the liquid form or the gel form is preferable. In addition, a polymer structure thereof is also not particularly limited and may have a straight-chain structure or a branch structure. Specifically, a chain polymer, a cyclic polymer, a star polymer, a comb polymer, a brush polymer, a crosslinked polymer, and any combination thereof can be used. Among the polymers, a polyurethane resin having no crosslinked structure is preferable from the viewpoint of good compatibility with another component.

A weight-average molecular weight (Mw) of the component (A) is not particularly limited and is preferably 5,000 or more, 10,000 or more, 20,000 or more, 30,000 or more, 40,000 or more, 45,000 or more, 50,000 or more, 55,000 or more, or 60,000 or more, and preferably 150,000 or less, 100,000 or less, or 80,000 or less. Within such a range, adhesion durability and removability can be further improved. Note that, in this specification, as the weight-average molecular weight (Mw), a value measured by gel permeation chromatography (GPC) using polystyrene as a standard substance is used.

The component (A) content is not particularly limited and is preferably 5 mass % or more, 10 mass % or more, 15 mass % or more, 18 mass % or more, 20 mass % or more, or 22 mass % or more with respect to the total amount of the composition and is preferably 70 mass % or less, 50 mass % or less, 45 mass % or less, 41 mass % or less, 39 mass % or less, 35 mass % or less, 32 mass % or less, or 30 mass % or less. Within such a range, adhesion durability and removability can be further improved while the composition has excellent operability to enhance applicability.

### [(B) Polymerizable Compound Having at Least Two Polymerizable Functional Groups in One Molecule]

The component (B) that is a polymerizable compound having at least two polymerizable functional groups in one molecule in the artificial nail composition of the present invention is a substance that reacts by external energy such as light or heat and forms a polymer. The component (B) imparts adhesion durability and removability to the artificial nail composition of the present invention. The component (B) is not particularly limited as long as the component (B) is a compound having at least two ethylenically unsaturated double bonds as the polymerizable functional groups in one molecule, and a known polymerizable compound can be used. Specific examples of the ethylenically unsaturated group include a (meth)acryloyl group, a (meth)acryloyloxy group, a (meth)acrylamide group, an isoprenyl group, a vinyl group, a vinyl ether group, a methyl vinyl ether group, an allyl group, an allyl ether group, and a maleimide group and are not limited thereto. One or two or more of ethylenically unsaturated groups may be contained. Among the ethylenically unsaturated groups, from the viewpoint of curability, surface hardness, and adhesion durability, at least one selected from the group consisting of a (meth)acryloyl group, a (meth)acryloyloxy group, and an isoprenyl group is preferable.

Note that, in this specification, the term, (meth)acryloyl, encompasses both acryloyl and methacryloyl, the term, (meth)acrylate, encompasses both acrylate and methacrylate, the term, (meth)acryloyloxy, encompasses both acryloyloxy and methacryloyloxy, and the term, (meth)acrylamide, encompasses both acrylamide and methacrylamide.

Specific examples of the component (B) having two ethylenically unsaturated groups in one molecule include polymerizable monomers such as urethane di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, triethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(meth)acrylate, propoxylated ethoxylated bisphenol A di(meth)acrylate, tricyclodecane dimethanol di(meth)acrylate, isoprenyl (meth)acrylate, and bis[2-(methacryloyloxy)ethyl]phosphate; an oligomer of the polymerizable monomers; and a polymer of the polymerizable monomers and are not limited thereto. The oligomer and the polymer may contain one or two or more of the examples of polymerizable monomers.

Specific examples of the component (B) having three or more ethylenically unsaturated groups in one molecule include polymerizable monomers such as urethane tri(meth)acrylate, urethane tetra(meth)acrylate, urethane penta(meth)acrylate, urethane hexa(meth)acrylate, trimethylolpropane tri(meth)acrylate, ethoxylated trimethylolpropane tri(meth)acrylate, ethoxylated glycerin tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, ethoxylated isocyanuric acid tri(meth)acrylate, pentaerythritol tetra(meth)acrylate, tetramethylolmethane tetra(meth)acrylate, dipentaerythritol tetra(meth)acrylate, ditrimethylolpropane tetra(meth)acrylate, dipentaerythritol penta(meth)acrylate, caprolactone-modified pentaerythritol penta(meth)acrylate, dipentaerythritol hexa(meth)acrylate, and caprolactone-modified pentaerythritol hexa(meth)acrylate; an oligomer of the polymerizable monomers; and a polymer of the polymerizable monomers and are not limited thereto. The oligomer and the polymer may contain one or two or more of the examples of polymerizable monomers.

A molecular weight of a monomer of the component (B) is not particularly limited and is preferably less than 1,000. A weight-average molecular weight (Mw) of an oligomer of the component (B) is not particularly limited and is preferably 200 to 30,000 and more preferably 700 to 10,000. A weight-average molecular weight (Mw) of a polymer of the component (B) is not particularly limited and is preferably more than 30,000. Within the range, the operability, the adhesion durability, and the removability can be further improved.

A monomer of the component (B) is a liquid under a 23° C. atmosphere and preferably has fluidity. Specifically, a viscosity at 23° C. and at a shear rate of 10 s⁻¹ measured using a rheometer which is a dynamic viscoelasticity measuring device is preferably 20,000 mPa·s or less, more preferably 10,000 mPa·s or less, still more preferably 5,000 mPa·s or less, and particularly preferably 1,000 mPa·s or less. The oligomer of the component (B) may or may not have fluidity under the 23° C. atmosphere. Specifically, the viscosity at 23° C. and at the shear rate of 10 s⁻¹ measured using the rheometer which is the dynamic viscoelasticity measuring device is preferably 7,000 mPa·s or more, more preferably 7,000 to 3,000,000 mPa·s, still more preferably 7,000 to 2,500,000 mPa·s, and particularly preferably 7,000 to 2,200,000 mPa·s. The polymer of the component (B) may or may not have fluidity under the 23° C. atmosphere.

Two or more kinds of components (B) can be selected to make the artificial nail composition. Among the examples, it is preferable to use at least one selected from the group consisting of urethane di(meth)acrylate, ethylene glycol di(meth)acrylate, triethylene glycol di(meth)acrylate, polyethylene glycol di(meth)acrylate, ethoxylated bisphenol A di(meth)acrylate, propoxylated bisphenol A di(meth)acrylate, propoxylated ethoxylated bisphenol A di(meth)acrylate, isoprenyl (meth)acrylate, bis[2-(methacryloyloxy)ethyl]phosphate, urethane tri(meth)acrylate, urethane tetra(meth)acrylate, urethane penta(meth)acrylate, urethane hexa(meth)acrylate, trimethylolpropane tri(meth)acrylate, ethoxylated trimethylolpropane tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, ethoxylated isocyanuric acid tri(meth)acrylate, pentaerythritol tetra(meth)acrylate, tetramethylolmethane tetra(meth)acrylate, dipentaerythritol tetra(meth)acrylate, ditrimethylolpropane tetra(meth)acrylate, dipentaerythritol penta(meth)acrylate, caprolactone-modified pentaerythritol penta(meth)acrylate, dipentaerythritol hexa(meth)acrylate, caprolactone-modified pentaerythritol hexa(meth)acrylate, an oligomer thereof, and a polymer thereof.

In addition, the component (B) is preferably a compound having two or three ethylenically unsaturated double bonds as polymerizable functional groups in one molecule. That is, among the examples, it is preferable to use at least one selected from the group consisting of urethane di(meth)acrylate, ethylene glycol di(meth)acrylate, triethylene glycol di(meth)acrylate, ethoxylated bisphenol A di(meth)acrylate, propoxylated bisphenol A di(meth)acrylate, propoxylated ethoxylated bisphenol A di(meth)acrylate, isoprenyl (meth)acrylate, bis[2-(methacryloyloxy)ethyl]phosphate, urethane tri(meth)acrylate, trimethylolpropane tri(meth)acrylate, ethoxylated trimethylolpropane tri(meth)acrylate, pentaerythritol tri(meth)acrylate, ethoxylated isocyanuric acid tri(meth)acrylate, an oligomer thereof, and a polymer thereof, and it is more preferable to use at least one selected from the group consisting of urethane di(meth)acrylate, ethylene glycol di(meth)acrylate, triethylene glycol di(meth)acrylate, propoxylated bisphenol A di(meth)acrylate, isoprenyl (meth)acrylate, bis[2-(methacryloyloxy)ethyl]phosphate, urethane tri(meth)acrylate, trimethylolpropane tri(meth)acrylate, ethoxylated trimethylolpropane tri(meth)acrylate, pentaerythritol tri(meth)acrylate, ethoxylated isocyanuric acid tri(meth)acrylate, an oligomer thereof, and a polymer thereof, and it is particularly preferable to use at least one selected from the group consisting of isoprenyl (meth)acrylate and trimethylolpropane tri(meth)acrylate. By containing the component (B), the adhesion durability and the removability can be further improved.

The component (B) content is not particularly limited and is preferably 0.01 mass % or more, 0.1 mass % or more, 0.3 mass % or more, 0.4 mass % or more, or 0.5 mass % or more with respect to the total amount of the composition, and is preferably 15 mass % or less, 10 mass % or less, 7 mass % or less, 5 mass % or less, 4 mass % or less, 3 mass % or less, 2 mass % or less, 1 mass % or less, or 0.8 mass % or less with respect to the total amount of the composition. Within such a range, the adhesion durability and the removability can be further improved.

### [(C) Polymerization Initiator]

The component (C) that is a polymerization initiator is a compound for initiating a polymerization reaction of a polymerizable compound. A kind of component (C) is not particularly limited, and a known polymerization initiator can be used. Examples of the component (C) include a photopolymerization initiator that serves as a starting point of a polymerization reaction by irradiation with an energy ray such as a visible light ray, an ultraviolet ray, an X-ray, or an electron beam, a thermal polymerization initiator that serves as a starting point of a polymerization reaction by heating to a certain temperature or higher, and a chemical polymerization initiator of a type in which two or more agents that serve as starting points of a polymerization reaction are mixed by mixing with a specific substance, and it is preferable to use the photopolymerization initiator. Examples of the photopolymerization initiator include a radical polymerization initiator that generates a radical by irradiation with an energy ray, a cationic polymerization initiator that generates a cation, an anionic polymerization initiator that generates an anion, and it is preferable to use the radical polymerization initiator. Specific examples of the radical photopolymerization initiator include benzoin ethers, benzyl ketals, α-dialkoxyacetophenones, α-hydroxyalkylphenones, α-aminoalkylphenones, acylphosphine oxides, benzophenones, thioxanthones, and titanocenes and are not limited thereto. In addition, two or more of these polymerization initiators can be selected to make the artificial nail composition. Among the examples, it is preferable to use at least one selected from the group consisting of α-hydroxyalkylphenones and acylphosphine oxides.

Specific examples of the α-hydroxyalkylphenones include 1-hydroxycyclohexyl phenyl ketone, 2-hydroxy-2-methyl-1-phenyl-propan-1-one, and α-aminoalkylphenone. Specific examples of the acylphosphine oxides include 2,4,6-trimethylbenzoyldiphenylphosphine oxide, 2,4,6-trimethylbenzoylphenylethoxyphosphine oxide, and bis(2,4,6-trimethylbenzoyl)phenylphosphine oxide. Among the examples, it is preferable to use at least one selected from 2,4,6-trimethylbenzoyldiphenylphosphine oxide, 2,4,6-trimethylbenzoylphenylethoxyphosphine oxide, and bis(2,4,6-trimethylbenzoyl)phenylphosphine oxide.

The component (C) content is not particularly limited and is preferably 0.1 mass % or more, 1 mass % or more, 2 mass % or more, 3 mass % or more, or 4 mass % or more with respect to the total amount of the composition, and is preferably 15 mass % or less, 10 mass % or less, 8 mass % or less, 7 mass % or less, or 6 mass % or less with respect to the total amount of the composition. Within such a range, the adhesion durability and the removability can be further improved.

### [(D) Polymerizable Compound]

The artificial nail composition of the present invention can further contain a component (D) that is a polymerizable compound excluding the component (B). The component (D) has an ethylenically unsaturated group as a polymerizable functional group and is not particularly limited as long as the component (D) is a compound other than the component (B), and a known polymerizable compound can be used.

Specific examples of the component (D) include methacrylic acid, acrylic acid, urethane (meth)acrylate, methoxyethylene glycol (meth)acrylate, methoxydiethylene glycol (meth)acrylate, methoxypolyethylene glycol (meth)acrylate, ethoxyethylene glycol (meth)acrylate, ethoxydiethylene glycol (meth)acrylate, ethoxypolyethylene glycol (meth)acrylate, phenoxyethylene glycol (meth)acrylate, phenoxypolyethylene glycol (meth)acrylate, 2-hydroxy-3-phenoxypropyl (meth)acrylate, 2-(meth)acryloyloxyethyl succinate, 2-(meth)acryloyloxyethyl phthalate, 2-(meth)acryloyloxypropyl hexaphthalate, stearyl (meth)acrylate, methyl (meth)acrylate, ethyl (meth)acrylate, hydroxyethyl (meth)acrylate, hydroxypropyl (meth)acrylate, 3-chloro-2-hydroxypropyl (meth)acrylate, isobornyl (meth)acrylate, tetrahydrofurfuryl (meth)acrylate, 2-(meth)acryloyloxyethyl dihydrogen phosphate, 2-(meth)acryloyloxyethyl phenyl hydrogen phosphate, 10-(meth)acryloyloxydecyl dihydrogen phosphate, 6-(meth)acryloyloxyhexyl dihydrogen phosphate, 2-(meth)acryloyloxyethyl 2-bromoethyl hydrogen phosphate, methylol (meth)acrylamide, dimethyl (meth)acrylamide, (meth)acryloylmorpholine, vinyl chloride, an oligomer thereof, and a polymer thereof, and are not limited thereto. These examples may be used singly or in combination of two or more kinds thereof.

A molecular weight of a monomer of the component (D) is not particularly limited and is preferably less than 1,000. A weight-average molecular weight (Mw) of an oligomer of the component (D) is not particularly limited and is preferably 200 to 30,000 and more preferably 700 to 10,000. A weight-average molecular weight (Mw) of a polymer of the component (D) is not particularly limited and is preferably more than 30,000. Within the range, the durability and the removability can be further improved.

A monomer of the component (D) is a liquid under a 23° C. atmosphere and preferably has fluidity. Specifically, a viscosity at 23° C. and at a shear rate of 10 s⁻¹ measured using a rheometer which is a dynamic viscoelasticity measuring device is preferably 20,000 mPa·s or less, more preferably 10,000 mPa·s or less, still more preferably 5,000 mPa·s or less, and particularly preferably 1,000 mPa·s or less. The oligomer of the component (D) may or may not have fluidity under the 23° C. atmosphere. Specifically, the viscosity at 23° C. and at the shear rate of 10 s⁻¹ measured using the rheometer which is the dynamic viscoelasticity measuring device is preferably 7,000 mPa·s or more, more preferably 7,000 to 3,000,000 mPa·s, still more preferably 7,000 to 2,500,000 mPa·s, and particularly preferably 7,000 to 2,200,000 mPa·s. The polymer of the component (D) may or may not have fluidity under the 23° C. atmosphere.

Two or more kinds of components (D) can be selected to make the artificial nail composition. Among the examples, it is preferable to use at least one selected from the group consisting of methacrylic acid, acrylic acid, urethane (meth)acrylate, methoxyethylene glycol (meth)acrylate, methoxydiethylene glycol (meth)acrylate, methoxypolyethylene glycol (meth)acrylate, ethoxyethylene glycol (meth)acrylate, ethoxydiethylene glycol (meth)acrylate, ethoxypolyethylene glycol (meth)acrylate, phenoxyethylene glycol (meth)acrylate, phenoxypolyethylene glycol (meth)acrylate, isobornyl (meth)acrylate, tetrahydrofurfuryl (meth)acrylate, hydroxyethyl (meth)acrylate, hydroxypropyl (meth)acrylate, acryloylmorpholine, an oligomer thereof, and a polymer thereof. In addition, from the viewpoint of improving both the adhesion durability and the removability, it is preferable to contain at least one selected from the group consisting of a polymerizable compound having a hydroxy group in a molecule, a polymerizable compound having an ether skeleton, an oligomer thereof, and a polymer thereof. In addition, it is particularly preferable to contain at least one of both a polymerizable compound having a hydroxy group in a molecule and a polymerizable compound having an ether skeleton. By containing the component (D), the adhesion durability and the removability can be further improved.

The component (D) content is not particularly limited, and the component (D) content is preferably 20 mass % or more, 30 mass % or more, 40 mass % or more, 50 mass % or more, or 60 mass % or more, and preferably 90 mass % or less, 80 mass % or less, 75 mass % or less, or 70 mass % or less, with respect to the total amount of the composition. Within such a range, the adhesion durability and the removability can be further improved while the composition has excellent operability to enhance the applicability.

### [Other Components]

The artificial nail composition of the present invention can contain components other than the components (A) to (D) as long as the effects of the present invention are not impaired. Examples of the other components include a supplement agent, an additive, a colorant, a leveling agent, a plasticizer, an antioxidant, a polymerization accelerator, a polymerization inhibitor, a coagulant, a preservative, a wax, a thickener, a fragrance, a UV blocking agent, a diffuser, a defoamer, a dispersant, a filler, a surfactant, a pigment, a dye, an excipient, an ion-releasing agent, an antibacterial agent, a chain transfer agent, and a silane coupling agent, which are widely used in artificial nail compositions. These examples may be used singly or in combination of two or more kinds thereof.

As the polymerization accelerator, a polyfunctional thiol compound having two or more thiol groups in one molecule can be used. By using the polyfunctional thiol compound, a curing reaction proceeds without being inhibited by oxygen. Examples of specific compounds as the polyfunctional thiol compounds include 1,2-ethanedithiol, 1,2-propanedithiol, 1,3-propanedithiol, 1,3-butanedithiol, 2,3-butanedithiol, 1,5-pentanedithiol, 1,6-hexanedithiol, 1,8-octanedithiol, 1,9-nonanedithiol, 1,10 decanedithiol, 1,2-benzenedithiol, 1,3-benzenedithiol, 1,4-benzenedithiol, 3,6-dichloro-1,2-benzenedithiol, toluene-3,4-dithiol, 1,5-naphthalenedithiol, ethylene glycol bis(thioglycolate), ethylene glycol bis(3-mercaptopropionate), 1,4-butanediol bisthioglycolate, tetraethylene glycol bis(3-mercaptopropionate), trimethylolpropane tris(thioglycolate), trimethylolpropane tris(3-mercaptopropionate), trimethylolpropane tris(3-mercaptobutyrate), tris[(3-mercaptopropionyloxy)-ethyl]isocyanurate, pentaerythritol tetrakis(thioglycolate), pentaerythritol tetrakis(3-mercaptopropionate), dipentaerythritol hexakis(3-mercaptopropionate), 1,4-bis(3-mercaptobutyryloxy)butane, pentaerythritol tetrakis(3-mercaptobutyrate), pentaerythritol tetrakis(3-mercaptobutyrate), 1,3,5-tris(3-mercaptobutyloxyethyl)-1,3,5-triazine-2,4,6(1H,3H,5H)-trione, dimercaptodiethylsulfide, 1,8-dimercapto-3,6-dithiaoctane, 1,2-bis[(2-mercaptoethyl)thio]-3-mercaptopropane, tetrakis(7-mercapto-2,5-dithiaheptyl)methane, trithiocyanuric acid, 1,2-benzenedimethane, thiol, 4,4′-thiobisbenzenethiol, 2-di-n-butylamino-4,6-dimercapto-s-triazine, 2,5-dimercapto-1,3,4-thiadiazole, 1,8-dimercapto-3,6-dioxaoctane, 1,5-dimercapto-3-thiapentane, tris(2-hydroxyethyl)isocyanurate trimercaptopropionate, 1,4-dimethylmercaptobenzene, 2,4,6-trimercapto-s-triazine, 2-(N,N-dibutylamino)-4,6-dimercapto-s-triazine, bis(4-(2-mercaptopropoxy)phenyl)methane, 1,1-bis(4-(2-mercaptopropoxy)phenyl)ethane, 2,2-bis(4-(2-mercaptopropoxy)phenyl) propane, 2,2-bis(4-(2-mercaptopropoxy)phenyl)butane, 1,1-bis(4-(2-mercaptopropoxy)phenyl)isobutane, 2,2-bis(4-(2-mercaptopropoxy)-3-methylphenyl)propane, 2,2-bis(4-(2-mercaptopropoxy)-5-methylphenyl)propane, bis(2-(2-mercaptopropoxy)-5-methylphenyl)methane, 2,2-bis(4-(2-mercaptopropoxy)-3-t-butylphenyl)propane, tris(4-(2-mercaptopropoxy)phenyl)methane, 1,1,1-tris(4-(2-mercaptopropoxy)phenyl)ethane, bis(4-(2-mercaptobutoxy)phenyl)methane, 2,2-bis(4-(2-mercaptobutoxy)phenyl)propane, tris(4-(2-mercaptobutoxy)phenyl)methane, 1,3,5-triazine-2,4,6-trithiol, and an alkyl vinyl ether adduct thereof. Here, the examples thereof are not limited thereto. These polymerization accelerators may be used singly or in combination of two or more kinds thereof.

As the polymerization accelerator, tertiary amines can also be used in addition to the polyfunctional thiol compounds. Specific examples of the tertiary amines include N,N-dimethylaniline, N,N-diethylaniline, N,N-di-n-butylaniline, N,N-dibenzylaniline, N,N-dimethyl-p-toluidine, N,N-dimethyl-m-toluidine, N,N-diethyl-p-toluidine, p-bromo-N,N-dimethylaniline, m-chloro-N,N-dimethylaniline, p-dimethylaminobenzaldehyde, p-dimethylaminoacetophenone, p-dimethylaminobenzoic acid, p-dimethylaminobenzoic acid ethyl ester, p-dimethylaminobenzoic acid amino ester, N,N-dimethylanthranilic acid methyl ester, N,N-dihydroxyethylaniline, N,N-dihydroxyethyl-p-toluidine, p-dimethylaminophenyl alcohol, p-dimethylaminostyrene, N,N-dimethyl-3,5-xylidine, 4-dimethylaminopyridine, N,N-dimethyl-α-naphthylamine, N,N-dimethyl-β-naphthylamine, tributylamine, tripropylamine, triethylamine, N-methyldiethanolamine, N-ethyldiethanolamine, N,N-dimethylhexylamine, N,N-dimethyldodecylamine, N,N-dimethylstearylamine, N,N-dimethylaminoethyl methacrylate, N,N-diethylaminoethyl methacrylate, and 2,2′-(n-butylimino)diethanol. Here, the examples thereof are not limited thereto. These polymerization accelerators may be used singly or in combination of two or more kinds thereof.

Some polymerization accelerators cause a polymerization reaction with the component (B) or the component (D). Such polymerization accelerators are regarded as polymerizable compounds even in a case where the polymerization accelerators do not have an ethylenically unsaturated group in a structure thereof. Therefore, the polymerization accelerator having at least two polymerizable functional groups in one molecule is regarded as the component (B), and the preferred component (B) content is calculated from a total amount of the component (B) and the polymerization accelerator having at least two polymerizable functional groups in one molecule.

Similarly, a polymerization accelerator (excluding the polymerization accelerator regarded as the component (B)) having a polymerizable functional group is regarded as the component (D), and the preferred component (D) content is calculated from a total amount of the component (D) and the polymerization accelerator (excluding the polymerization accelerator regarded as the component (B)) having the polymerizable functional group.

In a case where the polymerization accelerator is contained, the content thereof is not particularly limited and is preferably 4 mass % or more, more preferably 6 mass % or more, still more preferably 11 mass % or more, and particularly preferably 15 mass % or more, with respect to the total amount of the composition. On the other hand, the upper limit of the polymerization accelerator content is also not particularly limited and is preferably 80 mass % or less, more preferably 70 mass % or less, still more preferably 60 mass % or less, and particularly preferably 30 mass % or less, with respect to the total amount of the composition. By setting the polymerization accelerator content within the above range, properties such as the flexibility, the adhesion durability, the removability, the curability, the surface hardness, and the strength can be further improved, and good surface curability can be exhibited without being affected by polymerization inhibition due to oxygen.

The artificial nail composition of the present invention can be applied to a natural nail, a nail on which a coating is formed by the artificial nail composition, and a base material such as an artificial resin chip, a resin film and a resin sheet and is not limited thereto. In addition, examples of a method of application include methods of application using a brush, a sponge, a spray, an inkjet, an air knife, a roll, or the like and are not limited thereto.

The viscosity of the artificial nail composition of the present invention is not particularly limited, and the artificial nail composition preferably has fluidity under the 23° C. atmosphere from the viewpoint of operability. Specifically, the viscosity of the artificial nail composition of the present invention is preferably 1,000,000 mPa·s or less, 500,000 mPa·s or less, 100,000 mPa·s or less, 50,000 mPa·s or less, 25,000 mPa·s or less, 20,000 mPa·s or less, 15,000 mPa·s or less, or 10,000 mPa·s or less and is preferably 1 mPa·s or more, 10 mPa·s or more, 100 mPa·s or more, 500 mPa·s or more, 1,000 mPa·s or more, 2,000 mPa·s or more, or 3,000 mPa·s or more. Within such a range, the adhesion durability and the removability can be further improved while the artificial nail composition has operability to the extent that the composition is easy to handle. Note that the viscosity of the artificial nail composition of the present invention is a measured value at a shear rate of 10 s⁻¹ when the viscosity is measured under a condition of a shear rate of 0.1 to 100 s⁻¹ using a parallel plate PP20 by a rheometer (model: Physica MCR 301 manufactured by Anton Paar GmbH) as a dynamic viscoelasticity measuring device.

A tensile strength of the artificial nail composition of the present invention when cured and a tensile strength of an artificial nail (cured product) obtained from the artificial nail composition of the present invention are not particularly limited and are preferably 0.01 MPa or more, 0.1 MPa or more, or 0.3 MPa or more and preferably 20 MPa or less, 15 MPa or less, 10 MPa or less, 5 MPa or less, 3 MPa or less, 2 MPa or less, or 1 MPa or less from the viewpoint of the adhesion durability and the removability. Within such a range, the adhesion durability and the removability can be further improved. Note that the tensile strength is a value measured under a condition of a crosshead speed of 10 mm/min using an Instron universal testing machine (model: Instron 5943 type manufactured by Instron Corporation) after the artificial nail composition is cured by light irradiation for 20 seconds using a gel nail light (trade name: PRESTO LED Light manufactured by Nail Labo Corporation) to prepare a dumbbell-shaped test piece having a thickness of 1.9 mm, a total length of 28.6 mm, an inter-tab distance of 25.0 mm, a length of a parallel portion of 15.0 mm, a radius of a shoulder portion of 7.0 mm, and a width of the parallel portion of 2.0 mm, and the dumbbell-shaped test piece is left still all day and night.

A breaking strain of the artificial nail composition of the present invention when cured and a breaking strain of the artificial nail (cured product) obtained from the artificial nail composition of the present invention are not particularly limited and are preferably 30% or more, 50% or more, 100% or more, 150% or more, 200% or more, or 250% or more and preferably 2000% or less, 1500% or less, 1000% or less, 700% or less, 600% or less, 500% or less, or 450% or less from the viewpoint of the adhesion durability and the removability. Within such a range, the adhesion durability and the removability can be further improved. Note that the breaking strain is a measured value of a breaking strain obtained when the tensile strength is measured.

A loss tangent of the artificial nail composition of the present invention when cured and a loss tangent of the artificial nail (cured product) obtained from the artificial nail composition of the present invention are not particularly limited and are preferably 0.01 or more, 0.1 or more, 0.3 or more, and 0.5 or more and preferably 2 or less, 1.5 or less, 1 or less, and 0.8 or less from the viewpoint of the adhesion durability and the removability. Within such a range, the adhesion durability and the removability can be further improved. Note that the loss tangent is calculated by the following method. The artificial nail composition is cured by light irradiation for 20 seconds using the gel nail light (trade name: PRESTO LED Light manufactured by Nail Labo Corporation) to prepare a test piece having a thickness of 0.6 mm, a width of 10 mm, and an arbitrary length. Dynamic viscoelasticity measurement is performed using the prepared test piece in an arbitrary temperature range including 37° C. under conditions of a tensile mode, an inter-chuck distance of 20 mm, a strain of 0.1%, a frequency of 1 Hz, and a temperature increase rate of 3° C./min by a dynamic viscoelasticity measuring device DMA (model: Q800 manufactured by TA Instruments), and a loss tangent at 37° C. is calculated from the obtained values of storage modulus and loss modulus.

A glass transition temperature of the artificial nail composition of the present invention when cured and a glass transition temperature of the artificial nail (cured product) obtained from the artificial nail composition of the present invention are not particularly limited and are preferably −100° C. or higher, −50° C. or higher, −40° C. or higher, −30° C. or higher, or −20° C. or higher and preferably 100° C. or lower, 50° C. or lower, 30° C. or lower, 10° C. or lower, or 0° C. or lower from the viewpoint of the adhesion durability and the removability. Within such a range, the adhesion durability and the removability can be further improved. Note that the glass transition temperature is calculated from values of the storage modulus and the loss modulus obtained by performing dynamic viscoelasticity measurement in the same manner as the calculation of the loss tangent.

The kind of artificial nail composition of the present invention is not particularly limited, and examples thereof include nail polish, a gel nail, and an acrylic nail. The nail polish is a coating material for a nail which is called a nail lacquer, a nail enamel, a manicure, or the like and is a composition for producing a coating excellent in aesthetic appearance by drying a contained solvent. The gel nail is a material containing a resin component that is cured by an ultraviolet ray or a visible light ray and is a composition that forms a coating excellent in aesthetic appearance by being applied to a natural nail or an artificial nail and then cured by irradiation with the ultraviolet ray or the visible light ray. The acrylic nail is a powder-liquid type material containing a polymer bead and polymerizable monomers and is a composition in which polymerization of the polymerizable monomers is initiated and cured by a peroxide contained in the polymer bead after powder and a liquid are mixed. The acrylic nail is characterized in that the acrylic nail can not only be applied but also be produced in a layered-up form, and is mainly used for extending a nail in many cases. Among the kinds of artificial nail composition, it is preferable that the artificial nail composition of the present invention is the gel nail. With the gel nail, it is possible to provide the artificial nail composition having the adhesion durability to enable putting-on to be maintained for a certain period of time without any problem in daily life and the excellent removability to curb damage to a nail at the time of removal.

In the gel nail, a base layer, a color layer, and a topcoat layer are generally laminated in this order on a natural nail or a base material, and a coating is formed. An application method of the artificial nail composition of the present invention is not particularly limited, and any one layer of the base layer, the color layer, and the topcoat layer can be formed. The artificial nail composition of the present invention is preferably used as the base layer from the viewpoint of having the adhesion durability to enable putting-on to be maintained for a certain period of time without any problem in daily life and the excellent removability to curb damage to a nail at the time of removal.

Examples of the gel nail include a gel nail for a hand which is applied to a nail of a hand, a gel nail for a foot which is applied to a nail of a foot, and a gel nail for an animal which is applied to a claw of an animal. The use of the artificial nail composition of the present invention is not particularly limited, and the artificial nail composition can be used for any gel nail for a hand, a foot, an animal, or the like.

### EXAMPLES

Hereinafter, Examples and Comparative Examples of the present invention will be specifically described, but the present invention is not limited to these Examples.

### [Components Used for Preparation of Artificial Nail Composition]

The components used for preparation of artificial nail compositions of Examples and Comparative Examples are described below. As the weight-average molecular weight (Mw), a value measured using tetrahydrofuran as an eluent and polystyrene as a standard substance from gel permeation chromatography (GPC) performed by a GPC measurement system (trade name: Nexera GPC System manufactured by Shimadzu Corporation) and a column (trade name: Styragel HR manufactured by Waters Corporation) is used.

### [Component (A) That Is Polyurethane Resin]

PU1: Polyurethane resin 1 (the number of polymerizable functional groups: 0, polyether skeleton, weight-average molecular weight (Mw): 65,000)
PU2: Polyurethane resin 2 (the number of polymerizable functional groups: 0, polyether skeleton, weight-average molecular weight (Mw): 23,500)

### [Component (B) That Is Polymerizable Compound Having at Least Two Polymerizable Functional Groups in One Molecule]

UA1: Urethane acrylate oligomer (the number of polymerizable functional groups: 2, polyether skeleton, weight-average molecular weight: 27000)
TMPTA: trimethylolpropane triacrylate
IPEMA: isoprenyl methacrylate
EGDMA: ethylene glycol dimethacrylate
TEGDMA: triethylene glycol dimethacrylate
UDMA: urethane dimethacrylate
Bis-GMA: bisphenol A glycerate dimethacrylate
UDA: urethane diacrylate
Bis-MEP: bis[2-(methacryloyloxy)ethyl]phosphate
PETA: pentaerythritol tetraacrylate
DPEHA: dipentaerythritol hexaacrylate

### [Component (C) That is Polymerization Initiator]

HPK: 1-hydroxycyclohexyl phenyl ketone
TPO: 2,4,6-trimethylbenzoyldiphenylphosphine oxide
TPOL: 2,4,6-trimethylbenzoylphenylethoxyphosphine oxide
Bis-TPO: bis(2,4,6-trimethylbenzoyl)phenylphosphine oxide

### [Component (D) That is Polymerizable Compound]

PEGEA2: ethoxydiethylene glycol acrylate
PEGMMA2: methoxydiethylene glycol methacrylate
PEGMA9: methoxy nonaethylene glycol acrylate
PEGMA13: methoxy tridecaethylene glycol acrylate
HEMA: hydroxyethyl methacrylate
HPMA: hydroxypropyl methacrylate
IBA: isobornyl acrylate
IBMA: isobornyl methacrylate

### [Other Components]

DPMP: 2,4-diphenyl-4-methyl-1-pentene
TABPS: alkyl trimethyl dibutyl phosphate
IPA: isopropanol
WA: water

### [Preparation of Artificial Nail Composition]

The individual components were calculated according to respective blending ratios shown in Tables 1 to 8 and mixed until a uniform liquid is produced under atmospheric pressure using a rotation/revolution type mixer (trade name: ARV-310P manufactured by THINKY CORPORATION), thereby preparing artificial nail compositions of Examples and Comparative Examples.

An evaluation method of the artificial nail compositions of Examples and Comparative Examples is as follows. Note that an evaluation was performed under indoor LED lighting at a room temperature of 23±2° C. and a humidity of 50±10% unless otherwise specified.

### [Viscosity Measurement]

Regarding the artificial nail compositions of Examples and Comparative Examples, the viscosity measurement was performed under a condition of a shear rate of 0.1 to 100 s⁻¹ using a parallel plate PP20 by the rheometer (model: Physica MCR 301 manufactured by Anton Paar GmbH) as the dynamic viscoelasticity measuring device, and a measured value at a shear rate of 10 s⁻¹ was set as the viscosity.

### [Measurement of Tensile Strength and Breaking Strain]

The artificial nail compositions of Examples and Comparative Examples were cured by light irradiation for 20 seconds using the gel nail light (trade name: PRESTO LED Light manufactured by Nail Labo Corporation) to prepare dumbbell-shaped test pieces having a thickness of 1.9 mm, a total length of 28.6 mm, an inter-tab distance of 25.0 mm, a length of a parallel portion of 15.0 mm, a radius of a shoulder portion of 7.0 mm, and a width of the parallel portion of 2.0 mm. The prepared test pieces were left still all day and night, and then the tensile strength and the breaking strain were measured under the condition of a crosshead speed of 10 mm/min using the Instron universal testing machine (model: Instron 5943 type manufactured by Instron Corporation).

### [Measurement of Dynamic Viscoelasticity]

The artificial nail compositions of Examples and Comparative Examples were cured by light irradiation for 20 seconds using the gel nail light (trade name: PRESTO LED Light manufactured by Nail Labo Corporation) to prepare test pieces having a thickness of 0.6 mm, a width of 10 mm, and an arbitrary length. The dynamic viscoelasticity measurement was performed under conditions of a tensile mode, an inter-chuck distance of 20 mm, a strain of 0.1%, a frequency of 1 Hz, and a temperature increase rate of 3° C./min by the dynamic viscoelasticity measuring device DMA (model: Q800 manufactured by TA Instruments), and the storage modulus and the loss modulus at 37° C. were measured. In addition, the loss tangent at 37° C. and the glass transition temperature (Tg) were calculated from the obtained values of the storage modulus and the loss modulus.

### [Procedure Evaluation]

Regarding the artificial nail compositions of Examples and Comparative Examples, five manicurists certified by the Japan Nailist Association performed procedure evaluation according to the following method.

### <Procedure Method>

Step 1: Oil and dust of a nail were wiped off with a nail cleanser-soaked wipe.
Step 2: The artificial nail compositions of Examples or Comparative Examples were applied.
Step 3: Photopolymerization was carried out by light irradiation for 20 seconds by the gel nail light (trade name: PRESTO LED Light manufactured by Nail Labo Corporation).
Step 4: Gel nails (trade name: PRESTO brush on color gel 003 manufactured by Nail Labo Corporation) were applied.
Step 5: Step 3 was carried out again.
Step 6: Step 4 was carried out again.
Step 7: Step 3 was carried out again.
Step 8: Gel nails (trade name: PRESTO brush on top gel manufactured by Nail Labo Corporation) were applied.
Step 9: Step 3 was carried out again.
Step 10: A surface unpolymerized layer of a cured gel nail was wiped off with the nail cleanser-soaked wipe.
Step 11: In a state in which the artificial nail was put on, two weeks passed as usual.
Step 12: The artificial nail compositions of Examples or Comparative Examples were removed by gently taking off the compositions by fingers as if to peel off a seal.

In each step of the above procedure method, the following items were evaluated.

### [Operability]

In step 2, the operability felt by a practitioner was checked through a sensory evaluation, and evaluation results according to the following criteria were brought together, and a comprehensive evaluation was performed. When a score is 2 or higher from the comprehensive evaluation, the operability can be said to be good.
1: Viscosity is low, and application is difficult to perform.
2: Although the viscosity is slightly low, application can be performed without any problem.
3: Application is easy with moderate viscosity.
4: Although the viscosity is slightly high, application can be performed without any problem.
5: Viscosity is high, and application is difficult to perform.

### [Adhesion Durability]

In step 11, the states of the artificial nails put on nails were visually observed once or more a day, and evaluation results according to the following criteria were brought together, and a comprehensive evaluation was performed. When a score is 2 or higher from the comprehensive evaluation, the adhesion durability can be said to be good.
1: The artificial nail compositions completely peeled off from the nails within two weeks.
2: Although a gap was partially observed between the nail and the artificial nail composition within seven days after the procedure, peeling of the artificial nail composition was not observed until after two weeks.
3: Although a gap was partially observed between the nail and the artificial nail composition between eight to fourteen days after the procedure, peeling of the artificial nail composition was not observed until after two weeks.
4: Even after two weeks passed, no gap was observed between the nail and the artificial nail composition, and peeling of the artificial nail composition was not observed.

### [Abrasion Resistance]

In step 11, the states of the artificial nails put on nails were visually observed after two weeks passed, and evaluation results according to the following criteria were brought together, and a comprehensive evaluation was performed. When a score is 3 or higher from the comprehensive evaluation, the abrasion resistance can be said to be good.
1: The artificial nail composition completely peeled off from the nail within two weeks and therefore could not be evaluated.
2: Significant abrasion and partial chipping were observed, and the aesthetic appearance could not be maintained.
3: Although abrasion was observed, the aesthetic appearance was not significantly impaired, and the abrasion was acceptable.
4: Compared to general gel nails, a degree of abrasion was slightly higher, but a sufficient aesthetic appearance was maintained.
5: Abrasion was little observed, and the aesthetic appearance equivalent to that required for general gel nails was maintained.

### [Removability]

In step 12, the state of the nail after the artificial nail composition was removed was observed, and evaluation results according to the following criteria were brought together, and comprehensive evaluation was performed. When a score is 3 or higher from the comprehensive evaluation, the removability can be said to be good.
1: The artificial nail composition completely peeled off from the nail within two weeks and therefore could not be evaluated.
2: Obvious breaking was observed over a wide range.
3: Partial mild breaking was observed.
4: A change in surface texture such as roughness was partially observed.
5: No significant change was observed as compared with a state before the procedure.

TABLE 1

| | | Exam- | Exam- | Exam- | Exam- | Exam- | Exam- | Exam- | Exam- |
|---|---|---|---|---|---|---|---|---|---|
| | | ple | ple | ple | ple | ple | ple | ple | ple |
| | Abbreviation | 1 | 2 | 3 | 4 | 5 | 6 | 7 | 8 |
| Component A | PU1 | 31.7 | 30.2 | 28.4 | 26.4 | 24.2 | 21.6 | 18.6 | 15.1 |
| Component B | TMPTA | 0.5 | 0.6 | 0.6 | 0.6 | 0.7 | 0.7 | 0.8 | 0.8 |
| Component D | PEGEA2 | 31.8 | 33.4 | 35.6 | 37.8 | 40.2 | 43.1 | 46.5 | 50.5 |
| | HPMA | 10.6 | 11.2 | 11.8 | 12.6 | 13.4 | 14.4 | 15.5 | 16.8 |
| | IBA | 21.2 | 20.1 | 18.9 | 17.6 | 16.1 | 14.4 | 12.4 | 10.1 |
| Component C | TPO | 4.2 | 4.5 | 4.7 | 5.0 | 5.4 | 5.8 | 6.2 | 6.7 |

| | | | | | | | | |
|---|---|---|---|---|---|---|---|---|
| Total | 100.0 | 100.0 | 100.0 | 100.0 | 100.0 | 100.0 | 100.0 | 100.0 |
| Viscosity/Pa · s | 15 | 12 | 10 | 8 | 5 | 3 | 2 | 1 |
| Tensile strength/MPa | 1.0 | 1.0 | 0.8 | 0.6 | 0.9 | 0.7 | 0.8 | 0.5 |
| Breaking strain/% | 385 | 386 | 327 | 301 | 323 | 253 | 222 | 215 |
| Storage modulus/MPa | 0.08 | 0.07 | 0.10 | 0.09 | 0.10 | 0.06 | 0.07 | 0.05 |
| Loss modulus/MPa | 0.06 | 0.05 | 0.06 | 0.06 | 0.06 | 0.05 | 0.05 | 0.04 |
| Loss tangent | 0.75 | 0.71 | 0.60 | 0.67 | 0.60 | 0.83 | 0.71 | 0.70 |
| Glass transition temperature/° C. | −3 | −4 | −8 | −10 | −12 | −13 | −16 | −18 |
| Operability | 4 | 4 | 3 | 3 | 3 | 3 | 2 | 2 |
| Adhesion durability | 4 | 4 | 4 | 4 | 4 | 4 | 4 | 4 |
| Abrasion resistance | 5 | 5 | 5 | 5 | 5 | 5 | 5 | 5 |
| Removability | 5 | 5 | 5 | 5 | 5 | 5 | 5 | 5 |

TABLE 2

| | | Exam- | Exam- | Exam- | Exam- | Exam- | Exam- | Exam- | Exam- |
|---|---|---|---|---|---|---|---|---|---|
| | | ple | ple | ple | ple | ple | ple | ple | ple |
| | Abbreviation | 9 | 10 | 11 | 12 | 13 | 14 | 15 | 16 |
| Component A | PU1 | | | | 18.9 | 24.2 | 24.0 | 23.4 | 22.8 |
| | PU2 | 40.2 | 38.1 | 35.5 | 15.1 | | | | |
| Component B | TMPTA | 0.5 | 0.5 | 0.6 | 0.6 | 0.4 | 1.3 | 3.9 | 6.3 |
| Component D | PEGEA2 | 19.1 | 21.2 | 23.7 | 25.1 | 40.3 | 40.1 | 38.9 | 37.9 |
| | HPMA | 9.6 | 10.6 | 11.8 | 12.6 | 13.5 | 13.3 | 13.0 | 12.7 |
| | IBA | | | | 12.6 | 16.2 | 16.0 | 15.6 | 15.2 |
| | IBMA | 26.8 | 25.4 | 23.7 | 10.1 | | | | |
| Component C | TPO | 3.8 | 4.2 | 4.7 | 5.0 | 5.4 | 5.3 | 5.2 | 5.1 |

| | | | | | | | | |
|---|---|---|---|---|---|---|---|---|
| Total | 100.0 | 100.0 | 100.0 | 100.0 | 100.0 | 100.0 | 100.0 | 100.0 |
| Viscosity/Pa · s | 12 | 9 | 7 | 7 | 6 | 5 | 4 | 3 |
| Tensile strength/MPa | 2.1 | 1.7 | 2.0 | 1.2 | 0.5 | 1.0 | 0.7 | 1.2 |
| Breaking strain/% | 434 | 435 | 370 | 355 | 409 | 221 | 189 | 101 |
| Storage modulus/MPa | 0.24 | 0.43 | 0.67 | 0.22 | 0.11 | 0.16 | 0.21 | 0.34 |
| Loss modulus/MPa | 0.18 | 0.25 | 0.35 | 0.12 | 0.07 | 0.10 | 0.12 | 0.19 |
| Loss tangent | 0.75 | 0.58 | 0.52 | 0.55 | 0.64 | 0.63 | 0.57 | 0.56 |
| Glass transition temperature/° C. | 17 | 12 | 7 | 1 | −13 | −11 | −8 | −3 |
| Operability | 4 | 3 | 3 | 3 | 3 | 3 | 3 | 3 |
| Adhesion durability | 3 | 3 | 3 | 4 | 4 | 4 | 3 | 3 |
| Abrasion resistance | 3 | 3 | 3 | 4 | 5 | 5 | 5 | 5 |
| Removability | 4 | 5 | 5 | 5 | 5 | 5 | 5 | 4 |

TABLE 3

| | | Exam- | Exam- | Exam- | Exam- | Exam- |
|---|---|---|---|---|---|---|
| | | ple | ple | ple | ple | ple |
| | Abbreviation | 17 | 18 | 19 | 20 | 21 |
| Component A | PU1 | 24.2 | 24.2 | 24.0 | 23.4 | 22.8 |
| Component B | IPEMA | 0.4 | 0.7 | 1.3 | 3.9 | 6.3 |
| Component D | PEGEA2 | 40.3 | 40.2 | 40.1 | 38.9 | 37.9 |
| | HPMA | 13.5 | 13.4 | 13.3 | 13.0 | 12.7 |
| | IBA | 16.2 | 16.1 | 16.0 | 15.6 | 15.2 |
| Component C | TPO | 5.4 | 5.4 | 5.3 | 5.2 | 5.1 |

| | | | | | |
|---|---|---|---|---|---|
| Total | 100.0 | 100.0 | 100.0 | 100.0 | 100.0 |
| Viscosity/Pa · s | 6 | 5 | 5 | 3 | 3 |
| Tensile strength/MPa | 0.7 | 0.8 | 1.1 | 0.9 | 1.5 |
| Breaking strain/% | 470 | 307 | 253 | 188 | 121 |
| Storage modulus/MPa | 0.11 | 0.14 | 0.16 | 0.24 | 0.33 |
| Loss modulus/MPa | 0.07 | 0.09 | 0.10 | 0.13 | 0.18 |
| Loss tangent | 0.64 | 0.64 | 0.63 | 0.54 | 0.55 |
| Glass transition | −12 | −10 | −9 | −7 | −3 |
| temperature/° C. | | | | | |
| Operability | 3 | 3 | 3 | 3 | 3 |
| Adhesion durability | 4 | 4 | 4 | 3 | 3 |
| Abrasion resistance | 5 | 5 | 5 | 5 | 5 |
| Removability | 5 | 5 | 5 | 5 | 4 |

TABLE 4

| | | Exam- | Exam- | Exam- | Exam- | Exam- | Exam- | Exam- | Exam- |
|---|---|---|---|---|---|---|---|---|---|
| | | ple | ple | ple | ple | ple | ple | ple | ple |
| | Abbreviation | 22 | 23 | 24 | 25 | 26 | 27 | 28 | 29 |
| Component A | PU1 | 24.2 | 24.2 | 24.2 | 24.2 | 24.2 | 24.2 | 24.2 | 24.2 |
| Component B | EGDMA | 0.7 | | | | | | | |
| | TEGDMA | | 0.7 | | | | | | |
| | UDMA | | | 0.7 | | | | | |
| | Bis-GMA | | | | 0.7 | | | | |
| | UDA | | | | | 0.7 | | | |
| | Bis-MEP | | | | | | 0.7 | | |
| | PETA | | | | | | | 0.7 | |
| | DPEHA | | | | | | | | 0.7 |
| Component D | PEGEA2 | 40.2 | 40.2 | 40.2 | 40.2 | 40.2 | 40.2 | 40.2 | 40.2 |
| | HPMA | 13.4 | 13.4 | 13.4 | 13.4 | 13.4 | 13.4 | 13.4 | 13.4 |
| | IBA | 16.1 | 16.1 | 16.1 | 16.1 | 16.1 | 16.1 | 16.1 | 16.1 |
| Component C | TPO | 5.4 | 5.4 | 5.4 | 5.4 | 5.4 | 5.4 | 5.4 | 5.4 |

| | | | | | | | | |
|---|---|---|---|---|---|---|---|---|
| Total | 100.0 | 100.0 | 100.0 | 100.0 | 100.0 | 100.0 | 100.0 | 100.0 |
| Viscosity/Pa · s | 5 | 5 | 5 | 5 | 5 | 5 | 7 | 7 |
| Tensile strength/MPa | 0.9 | 0.5 | 0.9 | 0.8 | 0.6 | 0.5 | 0.5 | 0.6 |
| Breaking strain/% | 475 | 897 | 854 | 623 | 557 | 273 | 359 | 306 |
| Storage modulus/MPa | 0.14 | 0.17 | 0.14 | 0.11 | 0.10 | 0.15 | 0.27 | 0.42 |
| Loss modulus/MPa | 0.08 | 0.12 | 0.09 | 0.08 | 0.07 | 0.07 | 0.12 | 0.19 |
| Loss tangent | 0.57 | 0.71 | 0.64 | 0.73 | 0.70 | 0.47 | 0.44 | 0.45 |
| Glass transition temperature/° C. | −10 | −11 | −9 | −7 | −10 | −9 | −13 | −14 |
| Operability | 3 | 3 | 3 | 3 | 3 | 3 | 3 | 3 |
| Adhesion durability | 2 | 3 | 2 | 3 | 4 | 4 | 4 | 4 |
| Abrasion resistance | 4 | 4 | 4 | 4 | 4 | 4 | 3 | 4 |
| Removability | 5 | 5 | 5 | 5 | 4 | 3 | 3 | 3 |

TABLE 5

| | | Example | Example | Example | Example | Example | Example | Example |
|---|---|---|---|---|---|---|---|---|
| | Abbreviation | 30 | 31 | 32 | 33 | 34 | 35 | 36 |
| Component A | PU1 | 19.0 | 20.1 | 21.3 | 22.6 | 25.9 | 27.9 | 30.3 |
| Component B | TMPTA | 0.5 | 0.6 | 0.6 | 0.6 | 0.7 | 0.8 | 0.8 |
| Component D | PEGEA2 | 53.0 | 50.2 | 47.4 | 44.1 | 35.9 | 31.0 | 25.2 |
| | HPMA | 10.6 | 11.2 | 11.8 | 12.6 | 14.4 | 15.5 | 16.8 |
| | IBA | 12.7 | 13.4 | 14.2 | 15.1 | 17.3 | 18.6 | 20.2 |
| Component C | TPO | 4.2 | 4.5 | 4.7 | 5.0 | 5.8 | 6.2 | 6.7 |

| | | | | | | | |
|---|---|---|---|---|---|---|---|
| Total | 100.0 | 100.0 | 100.0 | 100.0 | 100.0 | 100.0 | 100.0 |
| Viscosity/Pa · s | 1 | 2 | 3 | 4 | 7 | 11 | 14 |
| Tensile strength/MPa | 0.4 | 0.4 | 0.7 | 0.6 | 0.8 | 0.7 | 1.1 |
| Breaking strain/% | 238 | 278 | 260 | 319 | 369 | 385 | 438 |
| Storage modulus/MPa | 0.05 | 0.06 | 0.06 | 0.07 | 0.08 | 0.10 | 0.07 |
| Loss modulus/MPa | 0.03 | 0.04 | 0.04 | 0.05 | 0.05 | 0.06 | 0.05 |
| Loss tangent | 0.60 | 0.67 | 0.67 | 0.71 | 0.63 | 0.60 | 0.71 |
| Glass transition temperature/° C. | −17 | −15 | −13 | −11 | −10 | −7 | −5 |
| Operability | 2 | 2 | 3 | 3 | 3 | 4 | 4 |
| Adhesion durability | 2 | 3 | 3 | 4 | 4 | 4 | 4 |
| Abrasion resistance | 3 | 3 | 4 | 5 | 5 | 5 | 5 |
| Removability | 5 | 5 | 5 | 5 | 5 | 5 | 5 |

TABLE 6

| | | Exam- | Exam- | Exam- | Exam- | Exam- | Exam- | Exam- | Exam- | Exam- |
|---|---|---|---|---|---|---|---|---|---|---|
| | | ple | ple | ple | ple | ple | ple | ple | ple | ple |
| | Abbreviation | 37 | 38 | 39 | 40 | 41 | 42 | 43 | 44 | 45 |
| Component A | PU1 | 24.2 | 24.2 | 24.2 | 24.2 | 26.7 | 25.9 | 22.6 | 21.3 | 19.0 |
| Component B | TMPTA | 0.7 | 0.7 | 0.7 | 0.7 | 0.7 | 0.7 | 0.6 | 0.6 | 0.5 |
| Component D | PEGEA2 | | | | 40.2 | 44.5 | 43.1 | 37.8 | 35.5 | 31.8 |
| | PEGMMA2 | 40.2 | | | | | | | | |
| | PEGMA9 | | 40.2 | | | | | | | |
| | PEGMA13 | | | 40.2 | | | | | | |
| | HEMA | | | | 13.4 | | | | | |
| | HPMA | 13.4 | 13.4 | 13.4 | | 4.4 | 7.2 | 18.9 | 23.7 | 31.8 |
| | IBA | 16.1 | 16.1 | 16.1 | 16.1 | 17.8 | 17.3 | 15.1 | 14.2 | 12.7 |
| Component C | TPO | 5.4 | 5.4 | 5.4 | 5.4 | 5.9 | 5.8 | 5.0 | 4.7 | 4.2 |

| | | | | | | | | | |
|---|---|---|---|---|---|---|---|---|---|
| Total | 100.0 | 100.0 | 100.0 | 100.0 | 100.0 | 100.0 | 100.0 | 100.0 | 100.0 |
| Viscosity/Pa · s | 5 | 7 | 9 | 5 | 11 | 8 | 3 | 2 | 1 |
| Tensile strength/MPa | 1.0 | 0.6 | 0.6 | 0.9 | 0.6 | 0.8 | 1.2 | 1.7 | 2.7 |
| Breaking strain/% | 332 | 294 | 314 | 305 | 258 | 291 | 359 | 429 | 537 |
| Storage modulus/MPa | 0.12 | 0.08 | 0.09 | 0.11 | 0.05 | 0.08 | 0.22 | 0.36 | 0.67 |
| Loss modulus/MPa | 0.08 | 0.05 | 0.06 | 0.06 | 0.03 | 0.05 | 0.14 | 0.22 | 0.41 |
| Loss tangent | 0.67 | 0.63 | 0.67 | 0.55 | 0.60 | 0.63 | 0.64 | 0.61 | 0.61 |
| Glass transition temperature/° C. | −3 | −15 | −19 | −10 | −20 | −17 | −7 | −1 | 11 |
| Operability | 3 | 3 | 3 | 3 | 4 | 3 | 3 | 2 | 2 |
| Adhesion durability | 3 | 3 | 2 | 4 | 3 | 4 | 4 | 3 | 2 |
| Abrasion resistance | 4 | 4 | 4 | 5 | 4 | 5 | 5 | 4 | 3 |
| Removability | 5 | 5 | 5 | 5 | 5 | 5 | 5 | 5 | 5 |

TABLE 7

| | | Exam- | Exam- | Exam- | Exam- | Exam- | Exam- | Exam- | Exam- | Exam- |
|---|---|---|---|---|---|---|---|---|---|---|
| | | ple | ple | ple | ple | ple | ple | ple | ple | ple |
| | Abbreviation | 46 | 47 | 48 | 49 | 50 | 51 | 52 | 53 | 54 |
| Component A | PU1 | 24.2 | 24.2 | 24.2 | 24.5 | 25.2 | 24.8 | 24.5 | 23.5 | 22.9 |
| Component B | TMPTA | 0.7 | 0.7 | 0.7 | 0.7 | 0.7 | 0.7 | 0.7 | 0.7 | 0.6 |
| Component D | PEGEA2 | 40.2 | 40.2 | 40.2 | 40.8 | 41.9 | 41.3 | 40.8 | 39.2 | 38.3 |
| | HPMA | 13.4 | 13.4 | 13.4 | 13.6 | 14.0 | 13.8 | 13.6 | 13.1 | 12.7 |
| | IBA | 16.1 | 16.1 | 16.1 | 16.3 | 16.8 | 16.6 | 16.3 | 15.7 | 15.3 |
| Component C | HPK | 2.7 | | | | | | | | |
| | TPO | 2.7 | 2.7 | | | 1.4 | 2.8 | 4.1 | 7.8 | 10.2 |
| | TPOL | | 2.7 | 5.4 | | | | | | |
| | Bis-TPO | | | | 4.1 | | | | | |

| | | | | | | | | | |
|---|---|---|---|---|---|---|---|---|---|
| Total | 100.0 | 100.0 | 100.0 | 100.0 | 100.0 | 100.0 | 100.0 | 100.0 | 100.0 |
| Viscosity/Pa · s | 5 | 6 | 5 | 5 | 6 | 6 | 6 | 5 | 6 |
| Tensile strength/MPa | 0.7 | 1.0 | 1.1 | 0.7 | 0.6 | 0.6 | 0.6 | 0.7 | 0.6 |
| Breaking strain/% | 341 | 290 | 332 | 340 | 325 | 297 | 334 | 306 | 322 |
| Storage modulus/MPa | 0.20 | 0.11 | 0.11 | 0.12 | 0.22 | 0.28 | 0.14 | 0.07 | 0.06 |
| Loss modulus/MPa | 0.12 | 0.08 | 0.07 | 0.08 | 0.13 | 0.15 | 0.09 | 0.05 | 0.05 |
| Loss tangent | 0.60 | 0.73 | 0.64 | 0.67 | 0.59 | 0.54 | 0.64 | 0.71 | 0.83 |
| Glass transition temperature/° C. | −14 | −11 | −11 | −15 | −17 | −14 | −15 | −15 | −17 |
| Operability | 3 | 3 | 3 | 3 | 3 | 3 | 3 | 3 | 3 |
| Adhesion durability | 4 | 4 | 4 | 4 | 3 | 4 | 4 | 4 | 4 |
| Abrasion resistance | 4 | 5 | 5 | 5 | 4 | 4 | 5 | 5 | 5 |
| Removability | 4 | 5 | 5 | 5 | 5 | 4 | 5 | 5 | 5 |

TABLE 8

| | | Exam- | Exam- | Exam- | Exam- | Comparative | Comparative |
|---|---|---|---|---|---|---|---|
| | Abbreviation | ple 55 | ple 56 | ple 57 | ple 58 | Example 1 | Example 2 |
| Component A | PU1 | 24.1 | 24.0 | 23.8 | 23.6 | | 24.4 |
| Component B | UA1 | | | | | 24.2 | |
| | TMPTA | 0.7 | 0.7 | 0.7 | 0.7 | 0.7 | |
| Component D | PEGEA2 | 40.2 | 40.0 | 39.8 | 39.2 | 40.2 | 40.5 |
| | HPMA | 13.4 | 13.3 | 13.2 | 14.4 | 13.4 | 13.5 |
| | IBA | 16.1 | 16.0 | 15.9 | 15.8 | 16.1 | 16.2 |
| Component C | TPO | 5.4 | 5.3 | 5.3 | 5.3 | 5.4 | 5.4 |
| Other | DPMP | 0.1 | 0.7 | 1.3 | | | |
| components | TABPS | | | | 0.87 | | |
| | IPA | | | | 0.08 | | |
| | WA | | | | 0.05 | | |

| | | | | | | |
|---|---|---|---|---|---|---|
| Total | 100.0 | 100.0 | 100.0 | 100.0 | 100.0 | 100.0 |
| Viscosity/Pa · s | 5 | 5 | 6 | 5 | 1 | 6 |
| Tensile strength/MPa | 0.9 | 0.9 | 0.8 | 0.8 | 0.9 | 0.6 |
| Breaking strain/% | 372 | 524 | 752 | 319 | 112 | 1771 |
| Storage modulus/MPa | 0.11 | 0.11 | 0.04 | 0.09 | 0.12 | 0.04 |
| Loss modulus/MPa | 0.07 | 0.07 | 0.03 | 0.06 | 0.04 | 0.06 |
| Loss tangent | 0.64 | 0.64 | 0.75 | 0.67 | 0.33 | 1.50 |
| Glass transition temperature/° C. | −12 | −10 | −12 | −12 | −11 | −2 |
| Operability | 3 | 3 | 3 | 3 | 2 | 3 |
| Adhesion durability | 4 | 4 | 2 | 4 | 2 | 1 |
| Abrasion resistance | 5 | 5 | 3 | 5 | 2 | 1 |
| Removability | 5 | 5 | 5 | 5 | 2 | 1 |

According to the present invention, it is possible to provide the artificial nail composition having the adhesion durability to enable putting-on to be maintained for a certain period of time without any problem in daily life and excellent removability to curb damage to a nail at the time of removal.

What is claimed is:

1. An artificial nail composition comprising:
a component (A) that is a polyurethane resin;
a component (B) that is a polymerizable compound having at least two polymerizable functional groups in one molecule; and
a component (C) that is a polymerization initiator.

2. The artificial nail composition according to claim 1, wherein the component (A) that is a polyurethane resin has a polyether skeleton.

3. The artificial nail composition according to claim 1, wherein a weight-average molecular weight (Mw) of the component (A) that is a polyurethane resin is 5000 or more.

4. The artificial nail composition according to claim 1, wherein the component (B) that is a polymerizable compound having at least two polymerizable functional groups in one molecule has at least one selected from the group consisting of a (meth)acryloyl group, a (meth)acryloyloxy group, and an isoprenyl group.

5. The artificial nail composition according to claim 1, wherein the component (B) that is a polymerizable compound having at least two polymerizable functional groups in one molecule is a compound having two or three polymerizable functional groups in one molecule.

6. The artificial nail composition according to claim 1, wherein the component (C) that is a polymerization initiator is a photopolymerization initiator.

7. The artificial nail composition according to claim 1, wherein the component (C) that is a polymerization initiator is at least one selected from the group consisting of α-hydroxyalkylphenones and acylphosphine oxides.

8. The artificial nail composition according to claim 1, further comprising a component (D) that is a polymerizable compound excluding the component (B).

9. The artificial nail composition according to claim 8, wherein the component (D) that is a polymerizable compound has, in one molecule, at least one selected from the group consisting of a hydroxyl group and an ether skeleton.

10. The artificial nail composition according to claim 1, wherein the artificial nail composition contains, with respect to a total amount of the composition,
5 to 70 mass % of the component (A) that is a polyurethane resin,
0.01 to 15 mass % of the component (B) that is a polymerizable compound having at least two polymerizable functional groups in one molecule, and
0.1 to 15 mass % of the component (C) that is a polymerization initiator.

11. The artificial nail composition according to claim 8, wherein the artificial nail composition contains, with respect to a total amount of the composition,
5 to 70 mass % of the component (A) that is a polyurethane resin,
0.01 to 15 mass % of the component (B) that is a polymerizable compound having at least two polymerizable functional groups in one molecule,
0.1 to 15 mass % of the component (C) that is a polymerization initiator, and
20 to 90 mass % of the component (D) that is a polymerizable compound excluding the component (B).

12. The artificial nail composition according to claim 1, wherein the artificial nail composition has a tensile strength of 20 MPa or less when cured.

13. The artificial nail composition according to claim 1, wherein the artificial nail composition has a breaking strain of 30% or more when cured.

14. The artificial nail composition according to claim 1, wherein the artificial nail composition has a loss tangent of 0.01 or more when cured.

15. The artificial nail composition according to claim 1, wherein the artificial nail composition has a glass transition temperature of 100° C. or lower when cured.

16. An artificial nail obtained from the artificial nail composition according to claim 1.

17. The artificial nail according to claim 16, wherein the artificial nail has a tensile strength of 20 MPa or less.

18. The artificial nail according to claim 16, wherein the artificial nail has a breaking strain of 30% or more.

19. The artificial nail according to claim 16, wherein the artificial nail has a loss tangent of 0.01 or more.

20. The artificial nail according to claim 16, wherein the artificial nail has a glass transition temperature of 100° C. or lower.
